(19) United States
(12) Patent Application Publication
(10) Pub. No.: US 2026/0041342 A1
(43) Pub. Date: Feb. 12, 2026

(54) SAMPLE COLLECTION APPARATUS AND METHODS OF USE

(71) Applicant: University of Houston System, Houston, TX (US)

(72) Inventor: Huong Thi Phan, Houston, TX (US)

(21) Appl. No.: 18/998,901

(22) PCT Filed: Jul. 28, 2023
(86) PCT No.: PCT/US2023/071284

§ 371 Date: Jan. 27, 2025

Related U.S. Application Data
(60) Provisional application No. 63/369,745, filed on Jul. 28, 2022.

(51) Int. Cl. A61B 5/15

(52) U.S. Cl. CPC A61B 5/150022; A61B 5/150358

(57) ABSTRACT

Provided here are apparatuses and methods for sample collection. One such sample collection apparatus includes a stencil having a first element, a second element, and a hinge connecting the first element and the second element. The first element has a top layer, an optional middle layer, and a bottom layer bonded together compositely with one or more design cutouts traversing the layers. The second element has an optional top layer and a bottom layer. The apparatus further includes a removable compression clip configured to secure an adsorbent media to the stencil. The apparatus may include an optional rivet to attach the removable compression clip to the stencil in a movable manner around the rivet. Also provided is a method for collecting a plurality of samples using the sample collection apparatus provided herein.

### RELATED APPLICATIONS

This application claims priority to U.S. Provisional Application No. 63/369,745 filed on Jul. 28, 2022, the content of which is incorporated herein by reference in its entirety.

### BACKGROUND

Biological materials are routinely collected as samples for screening to detect disorders related to metabolic, hormonal, and blood problems. Some screening methods utilize the spotting of biological materials on absorbent media, which is sent to testing labs for analysis. If the quality of the spotting is found to be unsatisfactory by the testing lab, a repeat sampling must be performed. Issues such as excess sample, smearing of the sample, contamination of the sample from adjacent spotted samples can lead to unsatisfactory sampling.

Collection of capillary blood sample from a finger or heel stick is an alternative and less invasive method compared to blood obtained from the vein. Moreover, only a small amount of blood sample is obtained and applied to the filter paper through the dried blood spot (DBS) method. This method was first used in 1913 for blood glucose analysis and became well known with the detection of a metabolic disorder in newborns in 1960's. Since the year 2000, the DBS method is used in the general population to analyze more than 175 biomarkers including glucose, acylcarnitines, lipoproteins, C-reactive protein, vitamin A, zinc, cytokines, and antibodies for more than 30 viruses. This method may also be used to detect persistent organic pollutants such as pesticides and toxins. The DBS method has proven useful during the pandemic with the testing of SARS-CoV-2 antibody in the United States and Canada. This method provides a convenient way for patients to send their capillary blood sample in via self-collection kits which have paved the way for remote testing in several countries for tests such as the HIV viral load testing through the United Nations and World Health Organization.

Another problematic area for sampling of this type includes the newborn screen blood spot test. Newborn screening is a public health service that extends to nearly 4 million babies born annually in the United States. The newborn screening blood test started in 1960's, when Dr. Robert Guthrie developed a blood test that could screen for phenylketonuria, a metabolic disorder, in newborns. Currently, the test has the capability of screening for more than 60 disorders in the United States, with most of the States screening for 31 to 35 disorders, depending on each state's newborn screening panel. Some of the disorders included in the screening panel are related to metabolic, hormonal, and blood diseases. During the specimen collection process, the baby's normal response is to cry and vigorously move all extremities. This may cause the blood to smear and contaminate the blood spot card, resulting in completing another blood spot card or performing a second heel stick puncture in the same or another clinic visit. There remains a need to improve the quality of the spotted samples sent to testing labs to reduce re-spotting and delays in data analysis.

Newborn screening programs are also implemented in many other countries. The method of using capillary blood collected from the baby's heel stick and applying the sample to a blood spot card is very similar to the recommended standard practice in the U.S. In 2020, more than 40 European countries including Switzerland, Spain, Turkey, Ireland, France, Israel, Germany, Uzbekistan, etc. reported screening more than 90% of their newborns.

### SUMMARY

Provided here are apparatuses and methods to address these shortcomings of the art and provide other additional or alternative advantages. The disclosure herein provides one or more embodiments of apparatuses and methods for sample collection. In one aspect, a sample collection apparatus is provided. The apparatus includes a stencil having a first element, a second element, and a hinge connecting the first element and the second element. The first element has a top layer and a bottom layer, bonded together compositely with a plurality of design cutouts traversing the top layer and the bottom layer. The second element has a bottom layer. The sample collection apparatus also includes a removable compression clip configured to secure an adsorbent media to the stencil.

In another aspect, a sample collection apparatus is provided. The apparatus includes a stencil having a first element, a second element, and a hinge connecting the first element and the second element. The first element has a top layer, a middle layer, and a bottom layer, bonded together compositely with a plurality of design cutouts traversing the top layer, the middle layer, and the bottom layer. The second element has a top layer and a bottom layer. The sample collection apparatus also includes a removable compression clip configured to secure an adsorbent media to the stencil, and a fastener attaching the removable compression clip to the stencil in a movable manner around the fastener.

In some embodiments, the top layer of the first element has an absorbent paper for absorbing a sample and the bottom layer of the first element has a barrier layer for resisting leak and contamination. Some embodiments include the bottom layer of the second element having a partially or fully transparent, impervious layer. In some embodiments, the middle layer of the first element has a substantially inflexible porous paper cardstock. The top layer of the second element can include a barrier layer for resisting leak and contamination.

In some embodiments, the plurality of design cutouts produce a pattern to match an absorbent media sample collection pattern of a spotter card. In some embodiments, the hinge contains a plastic material or a paper cardstock. In some embodiments, the paper cardstock of the hinge or the middle layer of the first element is of about 130 pound basis weight (BSW).

In some embodiments, the removable compression clip is a single clip of a length equal to the hinge. In some embodiments, the removable compression clip secures a spotter card within the first element and the second element. In some embodiments, the removable compression clip is one or more of a slidable bar, a binder clip, a paper clip, or a clam clip.

Provided here are methods for collecting a plurality of biological samples. One such method includes the steps of inserting a spotter card having an absorbent media to receive the plurality of biological samples within a stencil between a first element of the stencil with a plurality of design cutouts and a second element of the stencil, the first element and the second element being connected by a hinge; securing the spotter card within the first element and the second element using a removable compression clip positioned at the hinge; spotting the plurality of biological samples on the absorbent media accessible through the design cutout. In certain embodiments, the method further includes, after the spotting step, determining saturation of the plurality of biological samples within the absorbent media via visual inspection through the second element and removing the removable compression clip from the stencil. The method may include the step of separating the spotter card with the plurality of biological samples from the stencil. The removable compression clip can be a slidable bar, a binder clip, a paper clip, or a clam clip.

In some embodiments, the first element includes a top layer and a bottom layer bonded together compositely with a plurality of design cutouts traversing the top layer and the bottom layer. In some embodiments, the second element includes a bottom layer. In some embodiments, the top layer of the first element includes an absorbent paper for absorbing a sample. In some embodiments, the bottom layer of the first element includes a barrier layer for resisting leak and contamination. In some embodiments. the bottom layer of the second element includes a partially or a fully transparent, impervious layer.

In some embodiments, the stencil further includes a middle layer in the first element and a top layer in the second element. The stencil may include a fastener attaching the removable compression clip to the stencil in a movable manner around the fastener. The middle layer of the first element can be a substantially inflexible porous paper cardstock. In some embodiments, the top layer of the second element includes a barrier layer for resisting leak and contamination. Examples of a fastener include a rivet, an anchor, a screw, or a nut and bolt. Examples of a rivet include a blind rivet, a semi-tubular rivet, a solid rivet, a split rivet, or a drive rivet.

In some embodiments, the plurality of design cutouts produce a pattern to match an absorbent media sample collection pattern of the spotter card. In some embodiments, the hinge can be a plastic material or a paper cardstock. In some embodiments, the paper cardstock of the hinge or the middle layer of the first element is of about 130 pound basis weight (BSW).

Aspects and advantages of these exemplary embodiments and other embodiments, are discussed in detail herein. Moreover, it is to be understood that both the foregoing information and the following detailed description provide merely illustrative examples of various aspects and embodiments, and are intended to provide an understanding of the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to illustrate embodiments of the disclosure more clearly.

FIG. 1 is a representation of the stencil with a removable compressive clip, according to an embodiment.

FIG. 2 is a representation of an end on view of the stencil showing the first element, second element and hinge, according to an embodiment.

FIG. 3 is a representation of a front view of the stencil highlighting the design cutouts, according to an embodiment.

FIG. 4 is a representation of an interior view of the stencil highlighting the transparent second element, according to an embodiment.

FIG. 5 is a representation of a bottom view of the stencil highlighting the second element with frosted transparent plastic layer, according to an embodiment.

FIG. 6 is a representation of a Texas issued newborn screen blood spot filter card, according to an embodiment.

FIG. 7 is a representation of an enlarged view of the absorbent media substrate attached to the Texas newborn screen blood spot filter card, according to an embodiment.

FIG. 8 is a representation of the positioning of the spotter card within a first element with design cutouts, a transparent second element, and a foldable hinge, according to an embodiment.

FIG. 9 is a representation of the insertion of the spotter card within the first element and transparent second element, positioned against the hinge, and alignment of the design cutouts with the pre-defined spotting locations of the spotter card, according to an embodiment.

FIG. 10 is a representation of securing the spotter card within the first element, the transparent second element, and the hinge using a removable compression clip positioned at the hinge, according to an embodiment.

FIG. 11 is a representation of the secured spotter card predetermined spotting locations aligned with the design cutouts and available for spotting with the biological material, according to an embodiment.

FIG. 12 is a representation of determination of the absorption of the biological material and saturation of the absorbent media, according to an embodiment.

FIG. 13 is a representation of the stencil with a removable compressive clip, according to an embodiment.

FIG. 14 is a representation of a front view of the stencil with a removable compressive clip and a blind rivet, according to an embodiment.

FIG. 15 is a representation of an end view of the stencil with the removable compressive clip swung away from the open, bi-fold stencil comprising the first element, second element, and hinge, according to an embodiment.

FIGS. 16-20 depict the process of inserting and securing the spotter card within the sample collection apparatus, according to an embodiment. FIGS. 16 and 17 depict removing the removable clip from the stencil and the removal clip, according to an embodiment. FIGS. 18-20 depict inserting and positioning the spotter card within the first element and transparent second element, positioned against the hinge, and alignment of the design cutouts with the pre-defined spotting locations of the spotter card, according to an embodiment.

FIG. 21 is a representation of a front view of the secured spotter card, with the predetermined spotting locations aligned with the design cutouts and available for spotting with the biological material, according to an embodiment.

FIG. 22 is a representation of a back view of the secured spotter card within the sample collection apparatus, which allows for determination of the absorption of the biological material and saturation of the absorbent media, according to an embodiment.

FIG. 23 schematically depicts the stencil that includes the first element, the second element, and the hinge, according to an embodiment.

### DETAILED DESCRIPTION

Presented here are an apparatus and methods to improve the quality of spotted samples, such as biological samples on an absorbent media substrate. The apparatus serves as a stencil encasing the absorbent media substrate and preventing excess biological material from contaminating the absorbent media substrate during the collection process. The apparatus can be used to collect any samples, including a biological sample, such as a body fluid sample (such as blood, plasma, serum, urine, cerebrospinal fluid, respiratory secretion, bronchoalveolar lavage fluid, gastrointestinal secretion, reproductive secretion, ascites, plural effusion), or a cytology sample obtained from animals, including a human adult, child, or infant. For example, the apparatus can be used in conjunction with the state-issued newborn screen blood spot filter card, which is provided to clinicians by the government, in the US and outside the US. Many countries (including the United States, Australia, United Kingdom, China, and Canada) and many states within the United States (such as Texas, Arkansas, Connecticut, Hawaii, Idaho, Illinois, Kentucky, Minnesota, Montana, Nebraska, Nevada, North Carolina, Oklahoma, Tennessee, Utah, and Washington) use newborn screen blood spot filter card of a similar format. Accordingly, the apparatus and methods provided herein can be used to collect samples on newborn screen blood spot filter cards issued by the state, federal government, other public institutions, or private institutions in various counties and states. For example, the apparatus and methods can be used in conjunction with the newborn screen blood spot filter card provided by the Texas Department of Health and Human Services. The purpose of this invention is to improve the quality of the capillary sample collected in newborns for the newborn screening blood spot test and reduce the time it takes for the health care personnel to collect the sample. Dried blood spot (DBS) sampling tests generally come in the form of a filter card and preprinted target circles for sample collection. In addition to newborn screening, this device would also work for any DBS sampling tests for infants and toddlers. The World Health Organization (WHO) recommends the use of DBS to collect specimen for diagnosis and monitoring human immunodeficiency virus (HIV), and Hepatitis B and C infections of infants in low-income settings.

The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “about” or “approximately” are defined as being close to as understood by one of ordinary skill in the art. In one non-limiting embodiment, the terms are defined to be within 10%, preferably within 5%, more preferably within 1%, and most preferably within 0.5%. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The term “plurality” as used herein refers to two or more items or components. The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

A sample collection apparatus is provided herein. Certain embodiments of the sample collection apparatus include a stencil having a first element, a second element, and a hinge connecting the first element and the second element. The first element has a first layer and a second layer bonded together compositely with one or more design cutouts traversing the first layer and the second layer. The second element has a transparent layer. Certain embodiments include a removable compression clip configured to secure an adsorbent media to the stencil. The first layer includes a substantially inflexible porous paper cardstock. In certain embodiments, the porosity of paper cardstock is matched to the fluidity of the biological sample being spotted to optimize the paper absorbency to contain excess biological material overflow. “Biological sample” or “biological material” as used herein refers to any sample or material obtained from a living organism, including a tissue sample and a body fluid sample. A tissue sample can include a biopsy sample or resection sample obtained from any parts of the body. A body fluid sample can include blood, plasma, serum, urine, cerebrospinal fluid, respiratory secretion, bronchoalveolar lavage fluid, gastrointestinal secretion, reproductive secretion, ascites, plural effusion, and any other fluid obtained from a living organism. In specific embodiments, a biological sample or a biological material includes a blood sample, for example, obtained from a newborn. In certain embodiments, the second layer includes a barrier layer. In certain embodiments, the design cutouts are selected to produce a pattern to match an absorbent media sample collection pattern of a spotting card. The apparatus may include where the transparent layer with an impervious layer.

In certain embodiments, the hinge includes a paper cardstock of at least 130 lb. weight. The paper card stock is cellulosic, nitro cellulosic, cotton fiber, or a combination thereof. In certain embodiments, the paper card stock of the hinge is configured with a crease, a thin section of paper stock, or a profile to assist in the uniform folding of the paper stock hinge. In certain embodiments, the hinge is connected to the first element and the second element on the outward facing surfaces, inward facing surfaces, or a combination thereof. The removable compression clip can include a single clip of a length equal to the hinge. The removable compression clip can secure a spotter card within the first element and the second element. In other embodiments, the removable compression clip includes one or more of a slidable bar, binder clip, paper clip, or clam clip In certain embodiments, the stencil is a bi-fold card with a double-layer top fold (first element) and single layer bottom fold (second element), that is attached together by a 130 pound basis weight (BSW) cardstock fold (hinge). In certain embodiments, the double-layer of the top fold consists of 100% cellulose paper (first layer) and glassine paper (second layer). The bottom fold is made of plastic PET (clear or frosty) material for visibility of saturation during the collection process. A slide bar is used to keep the tool in place and aligned over the filter card so that the five targeted circles are exposed during sample collection. Embodiments of the top layer of the first element are configured to absorb the excess blood during sample collection. In certain embodiments, this layer is made of high purity cotton-based cellulose paper for consistent absorption. This may be the same filter paper attached to the Newborn Screening Form (such as Texas Newborn Screening Form) (Whatman qualitative filter paper grade 0903) or any lab filter paper including sample carrier papers made from pure cotton linters without any additives that is intended for absorbing human bodily fluids.

In certain embodiments, the bottom layer of the first element contains glassine paper sheet. This sheet is a thin, semi-transparent, shiny paper made from bleached sulfite wood pulp. It is strong but flexible and ideal for resisting leaks and grease and helps prevents contamination. This layer protects the surface of the actual filter paper attached to the Newborn Screening Form (such as Texas Newborn Screening Form) during sample collection. This paper is also referred to as glassine weighing paper. In certain embodiments, the second layer is glassine paper, plastic film, or a pH neutral, acid free material that is resistant to moisture, air, and grease. In certain embodiments the pattern cut out of the top fold is one or more shapes of various dimensions. In certain embodiments, the top fold has five pre-punched 0.5 inch circles aligned in a horizontal fashion. In certain embodiments, the pattern of the cut outs is arranged in an order to match the desired sampling spots on the spotter card. In certain embodiments, the stencil is configured to accommodate spotter cards, blotters, or chromatographic media. In certain embodiments, the transparent second element is clear, translucent, or partially obscured. In certain embodiments, the spotting materials are chemicals, reference standards, and synthetic analogs of biological materials.

The second element has a single layer. In certain embodiments, this element is a translucent, frosty, or clear polypropylene plastic sheet of about 5-millimeter thickness. This allows visibility of the saturation of blood specimen and protects the bottom of the filter paper attached to the Newborn Screening Form (such as Texas Newborn Screening Form) during sample collection.

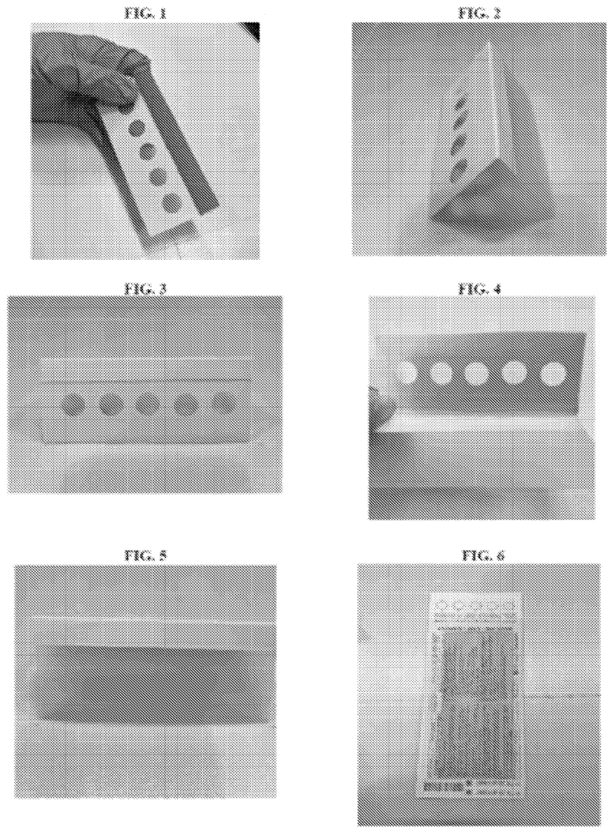

FIGS. 1-12 depict one embodiment of such a sample collection apparatus. FIG. 1 is a representation of the stencil with a removable compressive clip. This compressive clip is a slide bar. FIG. 2 is a representation of an end on view of the stencil showing the first element, the second element, and a hinge. FIG. 3 is a representation of a front view of the stencil highlighting the design cutouts. In an embodiment, the top layer is a 100% cellulose filter paper. FIG. 4 is a representation of an interior view of the stencil highlighting the transparent second element. In an embodiment, the bottom layer of the first element is made of glassine paper. FIG. 5 is a representation of a bottom view of the stencil highlighting the second element with frosted transparent plastic layer.

In some other embodiments, the sample collection apparatus includes a stencil having a first element, a second element, and a hinge connecting the first element and the second element. The first element has a top layer, a middle layer, and a bottom layer, bonded together compositely with a plurality of design cutouts traversing the top layer, the middle layer, and the bottom layer. The second element has a top layer and a bottom layer. The sample collection apparatus also includes a removable compression clip configured to secure an adsorbent media to the stencil, and a fastener attaching the removable compression clip to the stencil in a movable manner around the fastener. Examples of a fastener include a rivet, an anchor, a screw, or a nut and bolt. Examples of a rivet include a blind rivet, a semi-tubular rivet, a solid rivet, a split rivet, or a drive rivet.

In some embodiments, the top layer of the first element has an absorbent paper for absorbing a sample; the bottom layer of the first element has a barrier layer for resisting leak and contamination; and/or the bottom layer of the second element has a partially or fully transparent, impervious layer. In some embodiments, the middle layer of the first element has a substantially inflexible porous paper cardstock; and/or the top layer of the second element has a barrier layer for resisting leak and contamination.

In some embodiments, the plurality of design cutouts produce a pattern to match an absorbent media sample collection pattern of a spotter card. In some embodiments, the hinge contains a plastic material or a paper cardstock. In some embodiments, the paper cardstock of the hinge or the middle layer of the first element is of about 130 pound basis weight (BSW).

In some embodiments, the removable compression clip is a single clip of a length equal to the hinge. In some embodiments, the removable compression clip secures a spotter card within the first element and the second element. In some embodiments, the removable compression clip is one or more of a slidable bar, a binder clip, a paper clip, or a clam clip.

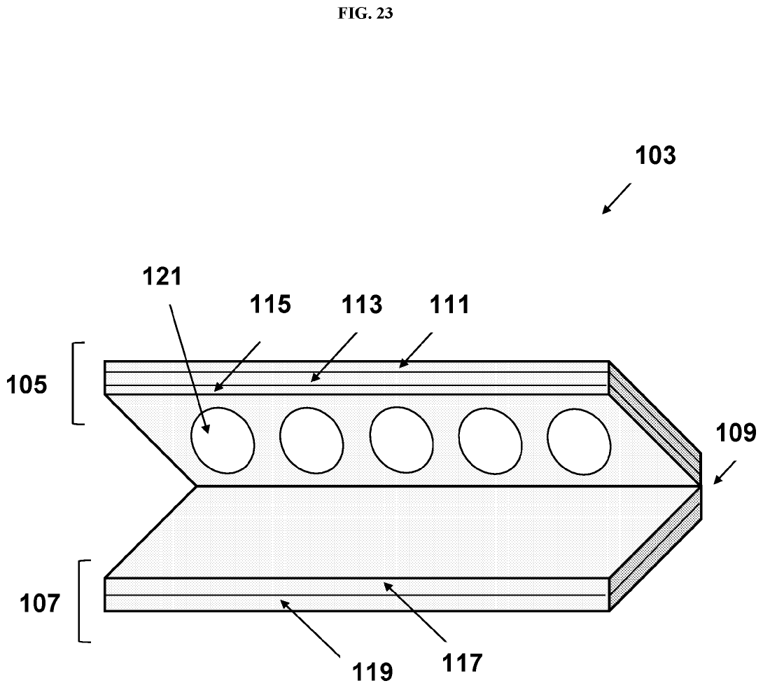

FIGS. 15 and 23 depict some embodiments of a sample collection apparatus provided herein. The example sample collection apparatus (101) includes a stencil (103) having a first element (105), a second element (107), and a hinge (109) connecting the first element (105) and the second element (107). The first element (105) has a top layer (111), an optional middle layer (113), and a bottom layer (115), bonded together compositely with a plurality of design cutouts traversing the top layer, the optional middle layer, and the bottom layer. The second element (107) has a top layer (117) and an optional bottom layer (119). The sample collection apparatus also includes a removable compression clip (123) configured to secure an adsorbent media to the stencil, and an optional blind rivet (125) attaching the removable compression clip to the stencil in a movable manner around the blind rivet.

The plurality of design cutouts (121) can produce a pattern to match an absorbent media sample collection pattern of a spotter card. The hinge (109) can contain a plastic material or a paper cardstock. The paper cardstock of the hinge (109) or the optional middle layer of the first element (113) can be of about 100-150 pound basis weight (BSW), such as about 100, 110, 120, 130, 140, or 150 pound BSW. In specific embodiments, the paper cardstock of the hinge (109) or the optional middle layer of the first element (113) has about 130 pound BSW.

The removable compression clip (123) can be a single clip of a length equal to the hinge (109). The removable compression clip (123) can secure a spotter card within the first element (105) and the second element (107). The removable compression clip (123) can be one or more of a slidable bar, a binder clip, a paper clip, or a clam clip.

As shown in FIG. 23, an example sample collection apparatus can include a stencil that is a bi-fold card (103). The bi-fold card can contain a triple-layer top fold (105) and double-layer bottom fold (107) attached together by a plastic hinge (109). The triple-layer of the top fold (105) can contain 100% cellulose paper (top layer, 111), 130 pound BSW cardstock paper (middle layer, 113), and glassine paper (bottom layer, 115). The top fold (103) can have five pre-punched 0.5 inch circles (121) aligned in a horizontal fashion. The double-layer of the bottom fold (107) can include glassine paper (top layer, 117) and a plastic PET (clear or frosty) material (bottom layer, 119) for visibility of saturation during the collection process. A C-shaped or oval-shaped plastic bar (123) is attached to the corner of the bottom fold by a blind rivet (125) to keep the tool in place and aligned over the filter card so that a plurality of targeted spots (such as 2, 3, 4, 5, 6, 7, 8, 9, 10, or more spots, such as circles) are exposed during sample collection.

For example, the top layer (111) of the first element can have an absorbent paper for absorbing a sample, and can be a filter paper layer that absorbs the excess blood during sample collection. The filter paper can be made of high purity cotton-based cellulose paper for consistent absorption. This may be the same filter paper attached to the Newborn Screening Form (such as Texas Newborn Screening Form) (Whatman qualitative filter paper grade 0903) or any lab filter paper including sample carrier papers made from pure cotton linters without any additives that is intended for absorbing human bodily fluids.

The optional middle layer (113) of the first element can have a substantially inflexible porous paper cardstock, for example a 130 pound WBS cardstock paper, placed between the top layer filter paper (111) and bottom layer glassine paper (115). The optional middle layer (113) provides support to strengthen the top fold.

The bottom layer (115) of the first element can contain a barrier layer for resisting leak and contamination, and can include glassine paper sheet that is a thin, semi-transparent, shiny paper made from bleached sulfite wood pulp. It is strong but flexible and ideal for resisting leaks and grease, and helps prevents contamination. This layer can protect the surface of the actual filter paper attached to the Newborn Screening Form (such as Texas Newborn Screening Form) during sample collection. This paper is also referred to as glassine weighing paper.

The optional top layer (117) of the second element can include a barrier layer for resisting leak and contamination, and can be a glassine paper sheet that is a thin, semi-transparent, shiny paper made from bleached sulfite wood pulp. It is strong but flexible and ideal for resisting leaks and grease, and helps prevents contamination. This layer protects the surface of the actual filter paper attached to the Newborn Screening Form (such as Texas Newborn Screening Form) during sample collection. This paper is also referred to as glassine weighing paper.

The bottom layer (119) of the second element can include a partially or fully transparent, impervious layer and can be a translucent, frosty, or clear polypropylene plastic sheet that is about 5 millimeter thickness. The translucency allows visual inspection from the bottom layer side of the saturation of the sample (such as blood specimen) and protects the bottom of the filter paper attached to the Newborn Screening Form (such as Texas Newborn Screening Form) during sample collection.

The attachment to the stencil (103) can include a C-shaped or oval-shaped plastic bar (123) that is attached to the top right corner of the bottom fold by a blind rivet (125). The swing clip may “swing” away to open the bi-fold tool (see FIG. 15) in order to wrap around the spotter card (such as state-issued newborn screen blood spot card) and “swing” back over the hinge part of the bi-fold tool to hold the blood spot card securely in place during capillary blood sample collection.

Embodiments also include methods for spotting of a plurality of biological samples. One such method the steps of inserting a spotter card having an absorbent media to receive the plurality of biological samples within a stencil between a first element of the stencil with a plurality of design cutouts and a second element of the stencil, the first element and the second element being connected by a hinge; securing the spotter card within the first element and the second element using a removable compression clip positioned at the hinge; spotting the plurality of biological samples on the absorbent media accessible through the design cutout. In further embodiments, the method further includes, after the spotting step, determining saturation of the plurality of biological samples within the absorbent media via visual inspection through the second element and removing the removable compression clip from the stencil. The method may include separating the spotter card with the plurality of biological samples from the stencil.

In some embodiments, the first element includes a top layer and a bottom layer bonded together compositely with a plurality of design cutouts traversing the top layer and the bottom layer. In some embodiments, the second element includes a bottom layer. In some embodiments, the top layer of the first element includes an absorbent paper for absorbing a sample. In some embodiments, the bottom layer of the first element includes a barrier layer for resisting leak and contamination. In some embodiments. the bottom layer of the second element includes a partially or a fully transparent, impervious layer.

In some embodiments, the stencil further includes a middle layer in the first element and a top layer in the second element. The stencil can optionally include a fastener attaching the removable compression clip to the stencil in a movable manner around the fastener. In some embodiments, the middle layer of the first element includes a substantially inflexible porous paper cardstock. In some embodiments, the top layer of the second element includes a barrier layer for resisting leak and contamination. Examples of a fastener include a rivet, an anchor, a screw, or a nut and bolt. Examples of a rivet include a blind rivet, a semi-tubular rivet, a solid rivet, a split rivet, or a drive rivet.

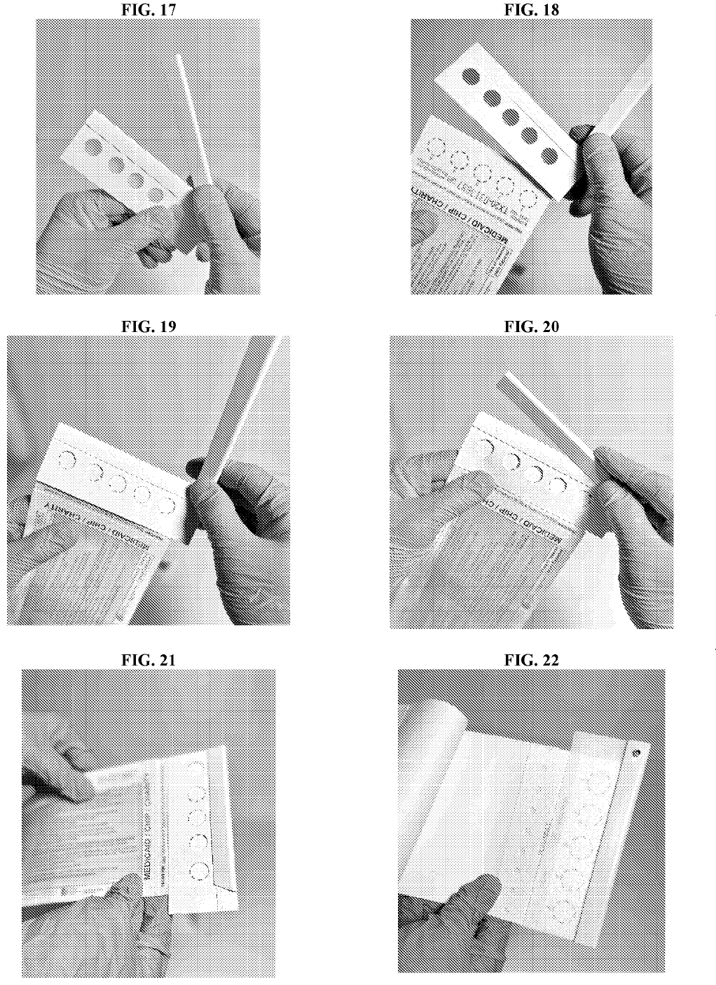

FIGS. 16-20 depict the process of inserting and securing the spotter card within the sample collection apparatus, according to an embodiment. FIGS. 16 and 17 depict removing the removable clip from the stencil and the removal clip. FIGS. 18-20 depict inserting and positioning the spotter card within the first element and transparent second element, positioned against the hinge, and alignment of the design cutouts with the pre-defined spotting locations of the spotter card. As shown in FIG. 21, the method allows to secure the spotter card within the sample collection apparatus, with the predetermined spotting locations of the spotter card aligned with the design cutouts and available to receive samples (such as biological samples). As shown in FIG. 22, the method allows to visually inspect, from the bottom layer side, the absorption of the sample (such as the biological material) and saturation of the absorbent media by the spotter card.

### EXAMPLE

It is mandatory for all newborns in the United States to undergo a screening unless the parent's refuse for religious reasons. During the specimen collection process, the baby's normal response is to cry and vigorously move all extremities. This may cause the blood to smear and contaminate the blood spot card, resulting in completing another blood spot card and/or performing a second heel stick puncture in the same clinic visit. Additionally, excess blood during the collection process may smear onto adjacent areas on the blood spot card and contaminate the sample, making it unsatisfactory by the newborn state lab.

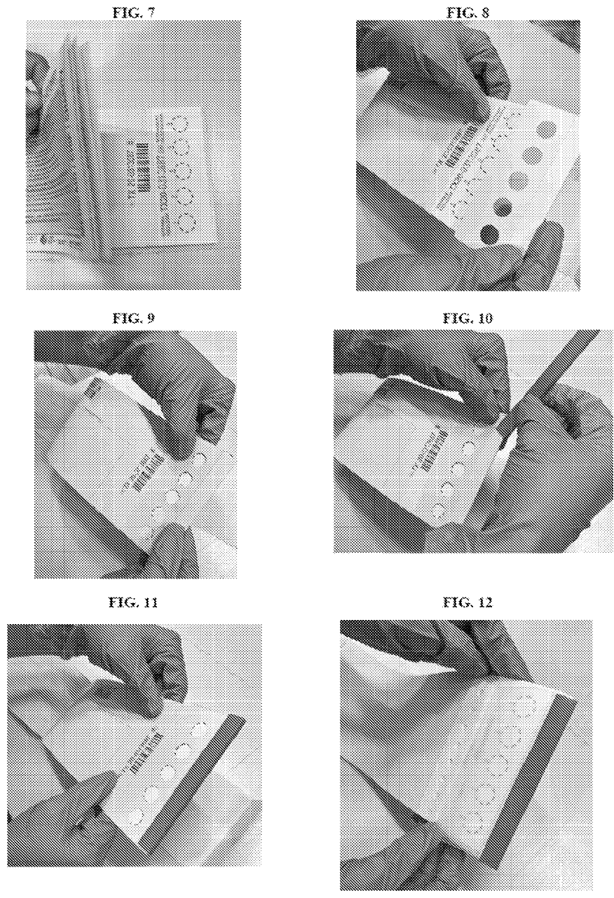

States issue state-specific newborn screen blood spot filter card (“card”) to clinicians. FIG. 6 is a screen blood spot filter issued by the Texas Department of Health and Human Services. The card contains a substrate section made from absorbent media to spot the biological material, in this case blood, as shown in FIG. 7. Placement of the apparatus over the substrate section of the card envelopes the substrate section within the stencil, and aligns the design cutouts of the stencil with the pre-defined spotting locations, as shown in FIG. 8 and FIG. 9. The combined apparatus and substrate section are secured in place by a compressive slide bar placed over the folded section of the stencil as shown in FIG. 10. Once secured, the pre-determined spotting locations are available for spotting with the biological material, FIG. 11. Five “clean” blood spots can be collected within the design cutouts of the stencil, FIG. 11. To ensure saturation of the absorbent media, the substrate section can be viewed from the transparent back of the stencil, FIG. 12. The stencil is removed after the spotting of the biological material and the card is sent to the testing lab for analysis.

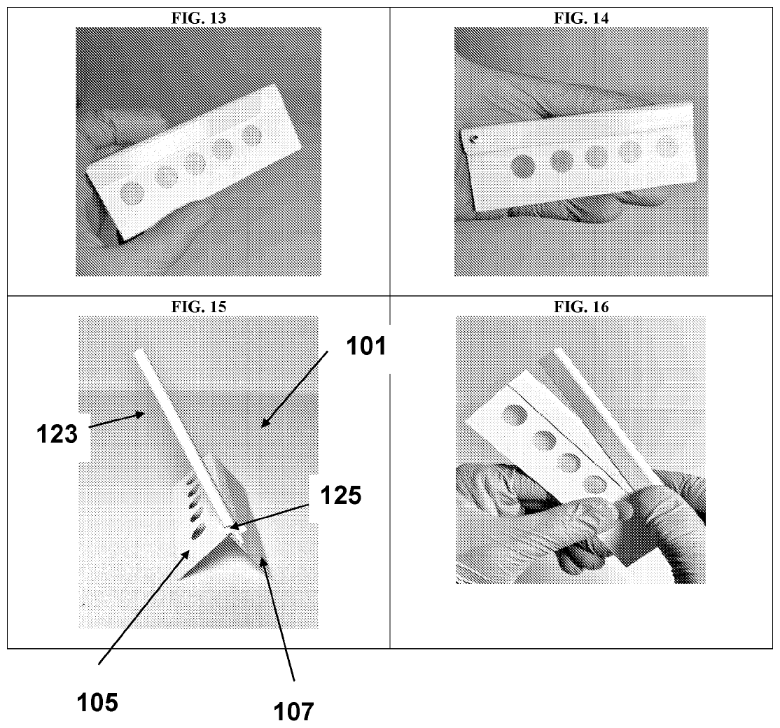

FIGS. 13-15 depict one embodiment of a sample collection apparatus. FIG. 13 is a representation of the stencil with a removable compressive clip. FIG. 14 is a representation of a front view of the stencil with a removable compressive clip and a blind rivet. FIG. 15 is a representation of an end view of the stencil with the removable compressive clip swung away from the open, bi-fold stencil comprising the first element, second element, and hinge.

FIGS. 16-20 depict the process of inserting and securing the spotter card within the sample collection apparatus. FIGS. 16 and 17 depict removing the removable clip from the stencil and the removal clip. FIGS. 18-20 depict inserting and positioning the spotter card within the first element and transparent second element, positioned against the hinge, and alignment of the design cutouts with the pre-defined spotting locations of the spotter card.

As shown in FIG. 21, the method allows to secure the spotter card within the sample collection apparatus, with the predetermined spotting locations of the spotter card aligned with the design cutouts and available to receive samples (such as biological samples). As shown in FIG. 22, the method allows to visually inspect, from the bottom layer side, the absorption of the sample (such as the biological material) and saturation of the absorbent media by the spotter card.

Other objects, features and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A sample collection apparatus, the apparatus comprising:
a stencil comprising a first element, a second element, and a hinge connecting the first element and the second element,
the first element comprising a top layer and a bottom layer bonded together compositely with a plurality of design cutouts traversing the top layer and the bottom layer, and
the second element comprising a bottom layer; and
a removable compression clip configured to secure an adsorbent media to the stencil.

2. A sample collection apparatus, the apparatus comprising:
a stencil having a first element, a second element, and a hinge connecting the first element and the second element,
the first element comprising a top layer, a middle layer, and a bottom layer bonded together compositely with a plurality of design cutouts traversing the top layer, the middle layer, and the bottom layer, and
the second element comprising a top layer and a bottom layer;
a removable compression clip configured to secure an adsorbent media to the stencil; and
a rivet attaching the removable compression clip to the stencil in a movable manner around the rivet.

3. The apparatus of claim 2, wherein:
the top layer of the first element includes an absorbent paper for absorbing a sample;
the bottom layer of the first element includes a barrier layer; and/or
the bottom layer of the second element includes a partially or fully transparent, impervious layer.

4. The apparatus of claim 2, wherein:
the middle layer of the first element includes a substantially inflexible porous paper cardstock; and/or
the top layer of the second element includes a barrier layer.

5. The apparatus of claim 2, wherein the plurality of design cutouts produce a pattern to match an absorbent media sample collection pattern of a spotter card.

6. The apparatus of claim 2, wherein the hinge is a plastic material or a paper cardstock.

7. The apparatus of claim 6, wherein the paper cardstock is of about 130 pound basis weight (BSW).

8. The apparatus of claim 2, wherein:
the removable compression clip is a single clip of a length equal to the hinge; and
the removable compression clip secures a spotter card within the first element and the second element.

9. A method for collecting a plurality of samples, the method comprising:
inserting a spotter card comprising an absorbent media to receive the plurality of samples within a stencil between a first element of the stencil with a plurality of design cutouts and a second element of the stencil, the first element and the second element being connected by a hinge;
securing the spotter card within the first element and the second element using a removable compression clip positioned at the hinge; and
spotting the plurality of samples on the absorbent media accessible through the design cutout.

10. The method of claim 9, further comprising, after the spotting step:
determining saturation of the plurality of samples within the absorbent media via visual inspection through the second element;
removing the removable compression clip from the stencil; and
optionally, separating the spotter card with the plurality of samples from the stencil.

11. The method of claim 9, wherein:
the first element comprises a top layer and a bottom layer bonded together compositely with a plurality of design cutouts traversing the top layer and the bottom layer; and
optionally, the second element comprises a bottom layer.

12. The method of claim 11, wherein:
the top layer of the first element comprises an absorbent paper;
the bottom layer of the first element comprises a barrier layer; and
optionally, the bottom layer of the second element comprises a partially or a fully transparent, impervious layer.

13. The method of claim 9, wherein the stencil further comprises:
a middle layer in the first element, the top layer, the middle layer and the bottom layer bonded together compositely with a plurality of design cutouts traversing the top layer, the middle layer, and the bottom layer;
a top layer in the second element; and
optionally, a rivet attaching the removable compression clip to the stencil in a movable manner around the rivet.

14. The method of claim 13, wherein the middle layer of the first element comprises a substantially inflexible porous paper cardstock, and optionally, the top layer of the second element comprises a barrier layer.

15. The method of claim 9, wherein the plurality of design cutouts produce a pattern to match an absorbent media sample collection pattern of the spotter card.
